(19) United States
(12) Patent Application Publication
(10) Pub. No.: US 2013/0284170 A1
(43) Pub. Date: Oct. 31, 2013

(54) SOUND-REDUCING TUBING IN A RESPIRATORY GAS DELIVERY SYSTEM

(71) Applicants: Khalid Said Mansour, Corona, CA (US); Brian W. Pierro, Yorba Linda, CA (US); Eric Porteous, Corona, CA (US); Geoff Wise, San Jose, CA (US)

(72) Inventors: Khalid Said Mansour, Corona, CA (US); Brian W. Pierro, Yorba Linda, CA (US); Eric Porteous, Corona, CA (US); Geoff Wise, San Jose, CA (US)

(21) Appl. No.: 13/460,589

(22) Filed: Apr. 30, 2012

(51) Int. Cl. A61M 16/00

(52) U.S. Cl. 128/204.18

(57) ABSTRACT

A system for reducing sound level in a respiratory gas delivery system is described. The system includes a first portion of a breathing circuit tubing and a second portion of the breathing circuit tubing positioned in-line with the first portion, wherein the second portion of the breathing circuit tubing is a sound-reducing tubing.

### FIELD OF THE INVENTION

The present technology relates generally to the respiratory field. More particularly, the present technology relates to respiratory gas delivery systems.

### BACKGROUND

In the field of respiratory therapy, it is known to provide a continuous positive airway pressure (CPAP) system and method for delivering continuous positive airway pressure, via the nasal cannula, to persons and some instances, to infants. This is particularly true in the case of prematurely born infants who frequently suffer with increased work of breathing due to immature lungs that have the propensity to collapse during exhalation and resist expansion during inhalation.

One particular method of treatment involves the use of nasal cannula that fits sealingly into the nares and is connected to a breathing system that generates a continuous flow of air with above atmospheric pressures, commonly referred to as continuous positive airway pressure (CPAP) therapy. The positive pressure is transmitted through the infant's airways and into the lungs, thereby preventing collapse during exhalation and augmenting expansion during inhalation.

There are a wide variety of devices in use for CPAP. The CPAP devices often include what is referred to as a generator body, which is essentially a housing forming a chamber that receives air pressure from tubing. The generator body typically has an exhalation port for air to escape during the exhalation phase, through exhalation tubing. Further, the generator body has a pair of nasal prongs which fit into the patient's nares to supply pressure into the nares.

### DESCRIPTION OF DRAWINGS

FIG. 1 shows a block diagram of a system for reducing sound level in a respiratory gas delivery system, in accordance with embodiments.

FIG. 2 shows a system for reducing sound level in a respiratory gas delivery system, in accordance with embodiments.

FIG. 3 shows a system for reducing sound level in a respiratory gas delivery system, in accordance with embodiments.

FIG. 4 shows various geometries of tubing, in accordance with embodiments.

FIG. 5 shows a flow chart of an example method for manufacturing a system for reducing sound level in a respiratory gas delivery system, in accordance with embodiments.

FIG. 6 shows a front perspective view of a patient breathing through a respiratory mask through the upper airways.

FIG. 7 shows a patient breathing with an endotracheal tube, wherein the patient's upper airways are bypassed.

FIG. 8 shows a flow diagram of a flow of gas during single limb ventilation.

FIG. 9 shows a flow diagram of a flow of gas during dual limb ventilation.

The drawings referred to in this description should not be understood as being drawn to scale unless specifically noted.

### DESCRIPTION OF EMBODIMENTS

Reference will now be made in detail to various embodiments, examples of which are illustrated in the accompanying drawings. While the subject matter will be described in conjunction with these embodiments, it will be understood that they are not intended to limit the subject matter to these embodiments. On the contrary, the subject matter described herein is intended to cover alternatives, modifications and equivalents, which may be included within the spirit and scope. Furthermore, in the following description, numerous specific details are set forth in order to provide a thorough understanding of the subject matter. However, some embodiments may be practiced without these specific details. In other instances, well-known structures and components have not been described in detail as not to unnecessarily obscure aspects of the subject matter.

### Overview of Discussion

Herein, various embodiments of a system for reducing sound level in a respiratory gas delivery system are described. The description begins with a brief general discussion of traditional respiratory gas delivery systems. This general discussion provides a framework of understanding for a more particularized description which follows, focusing on particular features and concepts of operation associated with one or more embodiments of the described system for reducing sound level.

### Respiratory Gas Delivery Systems

Traditional respiratory gas delivery systems for use in critical care and patient care settings typically involve a generator body, which is essentially a housing forming a chamber that receives air pressure from the tubing of a breathing circuit. The generator body typically has an exhalation port for air to escape during the exhalation phase, through exhalation tubing. Further, the generator body has a pair of nasal prongs that fit into the patient's nares to supply pressure into the nares.

Presently, the sound from the jets that are driven by the generator moves at least through the exhaust tubing, creating a significantly large amount of noise that is potentially harmful to the patient. For example, the high level of noise over extended periods may damage an infant's hearing or interrupt the sleep cycle, requiring it to expend greater energy which could otherwise be used for growth or development. The traditional device that is coupled with the respiratory gas delivery system and is used to reduce sound level is cumbersome, heavy, and/or at least partially occludes the breathing circuit's air pathway. Further, the traditional respiratory gas delivery system that includes a traditional device for reducing sound level encourages puddling within the air flow path, such that a patient's work of breathing increases during exhalation in order to push against the liquid buildup.

### Sound-Reducing Tubing in a Respiratory Gas Delivery System

As will be described in detail below, embodiments provide a system for reducing sound level in a respiratory gas delivery system and a method for manufacturing the system. For example, in one embodiment, the system includes a first portion of a breathing circuit tubing and a second portion of the breathing circuit tubing positioned in-line with the first portion, wherein the second portion is a sound-reducing tubing. The sound-reducing tubing is formed of material that has the effect of reducing the sound level moving there through. In one embodiment, the first portion is positioned at the patient-end of the respiratory gas delivery system.

Embodiments provide many benefits over traditional systems. For example, embodiments enable combining non-sound-reducing tubing (as the first portion) in-line with sound-reducing tubing within a breathing circuit. The non-sound-reducing tubing has unique and desired properties, such as flexibility, density, and compressibility, etc. Thus, embodiments enable sound level to be reduced by the sound-reducing tubing while allowing the non-sound reducing tubing to be located next to the patient-end such that it may be repositioned according to the patient's movement and environment.

Further, the sound-reducing tubing may be formed with differing geometries and/or materials. The resulting in-line combination of tubings, according to embodiments, creates a stronger breathing circuit that does not collapse as easily as the traditional system. Thus, when the patient moves around, the breathing circuit tubing of embodiments is able to adapt to the patient's position without becoming deformed and/or unusable. Additionally, in some embodiments, this combination of two different types of tubing assists in the reduction of moisture build-up.

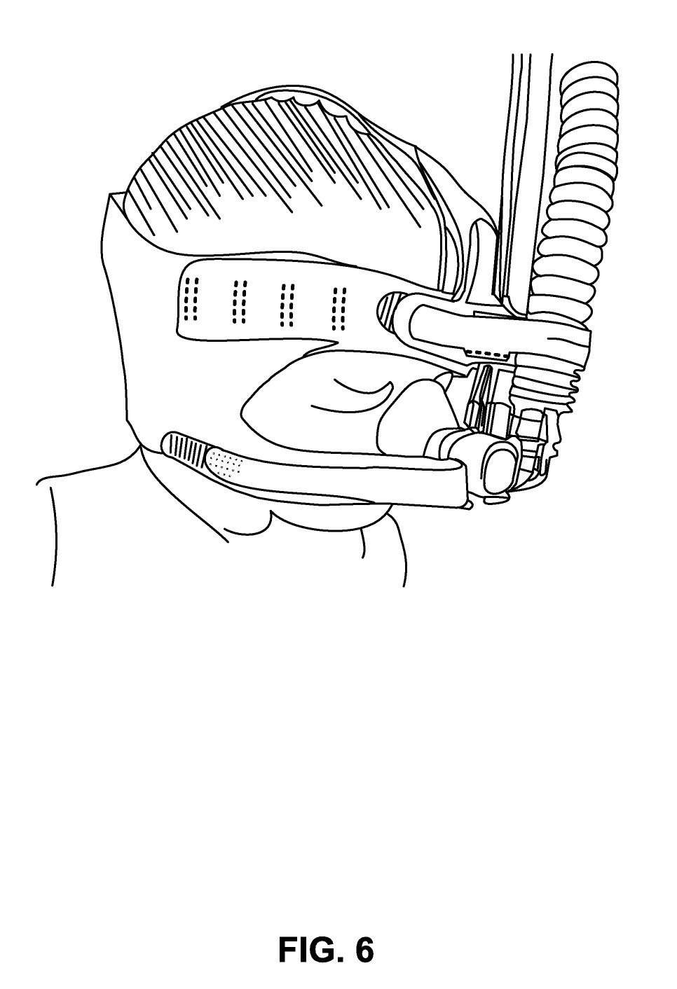

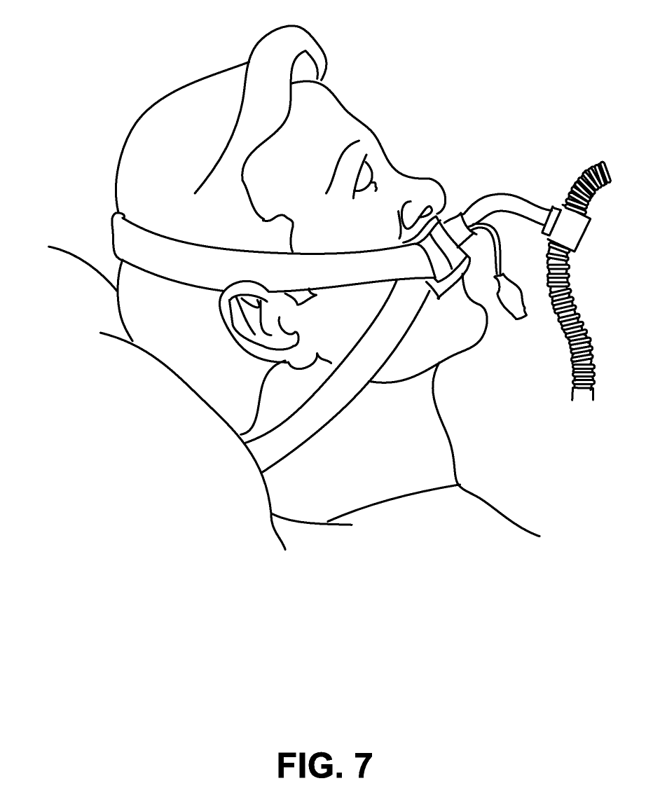

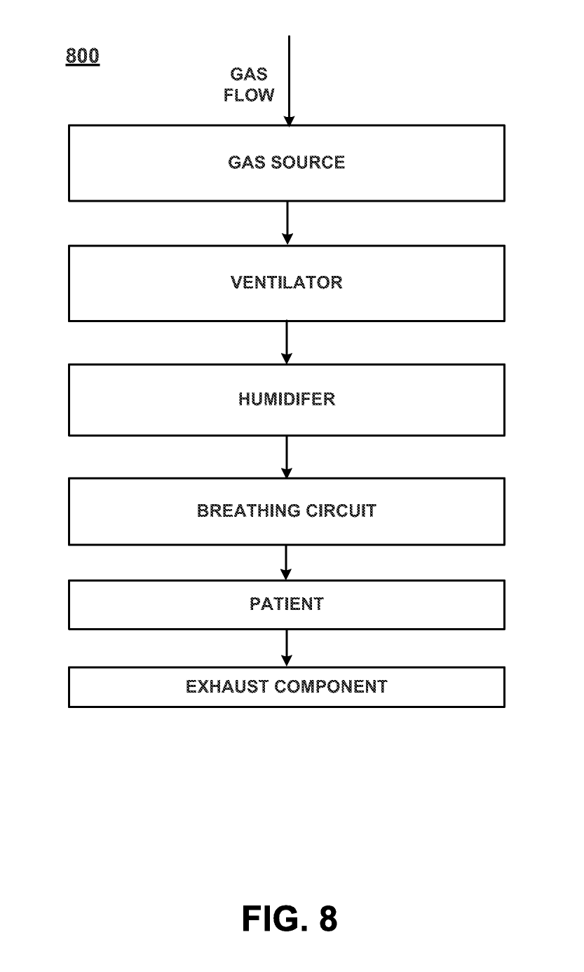

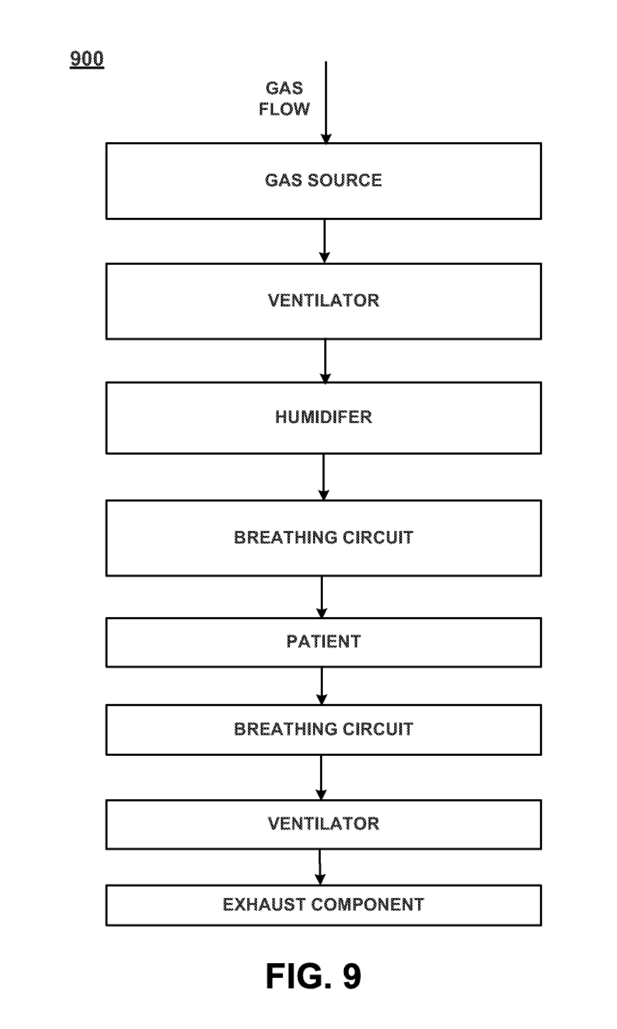

It should be noted that the methods and devices described herein may be used in various modes of respiratory care, including, but not limited to, non-invasive single limb ventilation, dual-limb invasive ventilation, dual-limb non-invasive ventilation, continuous positive airway pressure (CPAP), bubble CPAP, bi-level positive airway pressure (BiPAP), intermittent positive pressure (IPPB), bland aerosol therapy and oxygen therapy. In general, non-invasive single and dual-limb ventilation refers to the delivery of ventilator support using a mechanical ventilator, with one or multiple limbs, connected to a mask or mouthpiece instead of an endotracheal tube or tracheostomy interface. For example, FIG. 6 shows a front perspective view of a patient breathing with a mask through the upper airways (using a non-invasive ventilation system). A dual-limb invasive therapy refers to the delivery of ventilator support using a mechanical ventilator, with multiple limbs, connected to an endotracheal tube. For example, FIG. 7 illustrates a patient breathing with an endotracheal tube, wherein the patient's upper airways are bypassed (using an invasive ventilation system). Further, FIGS. 8 and 9 illustrate flow diagrams 800 and 900, respectively, of the flow of gas during single limb and dual limb ventilation, respectively. More particular, 800 of FIG. 8, with regards to single limb ventilation, shows gas flowing from a gas source to a ventilator, to a humidifier, to a breathing circuit, to a patient, to an exhaust component. In contrast, 900 of FIG. 9, with regards to dual limb ventilation, shows gas flowing from a gas source to a ventilator, to a humidifier, to a breathing circuit, to a patient, to a breathing circuit, to a ventilator, to an exhaust component.

CPAP refers to the maintenance of positive pressure in the airway throughout a respiratory cycle. Bubble CPAP refers to a procedure that doctors use to help promote breathing in premature newborns. In bubble CPAP, positive airway pressure is maintained by placing the expiratory limb of the circuit under water. The production of bubbles under the water produces a slight oscillation in the pressure waveform. BiPAP refers to the maintenance of a baseline positive pressure during inspiration and expiration, but with brief increases of this pressure periodically. IPPB refers to the non-continuous application of positive airway pressure when, for example, an episode of apnea is sensed. Bland aerosol therapy refers to the delivery of hypotonic, hypertonic, or isotonic saline, or sterile water in aerosolized form, to a patient as a medical intervention. Oxygen therapy refers to the delivery of oxygen to a patient, as a medical intervention.

The following discussion describes the architecture and operation of embodiments.

Breathing circuits are utilized to deliver such medical support as air and anesthetics from a machine that creates an artificial environment to a patient via tubes. Breathing circuits are used in surgical procedures, respiratory support and respiratory therapies. For example, in a most general case, breathing circuits include an inspiratory limb running from a ventilator to a patient and an expiratory limb running from the patient back to the ventilator.

The ventilator pushes gas through the inspiratory limb to reach the patient. The patient inhales this pushed gas and exhales gas into the expiratory limb. For purposes of the embodiments, any portion of the breathing circuit could be considered a patient circuit or conduit. It should be appreciated that embodiments are well suited to be used in any portion of the patient circuit.

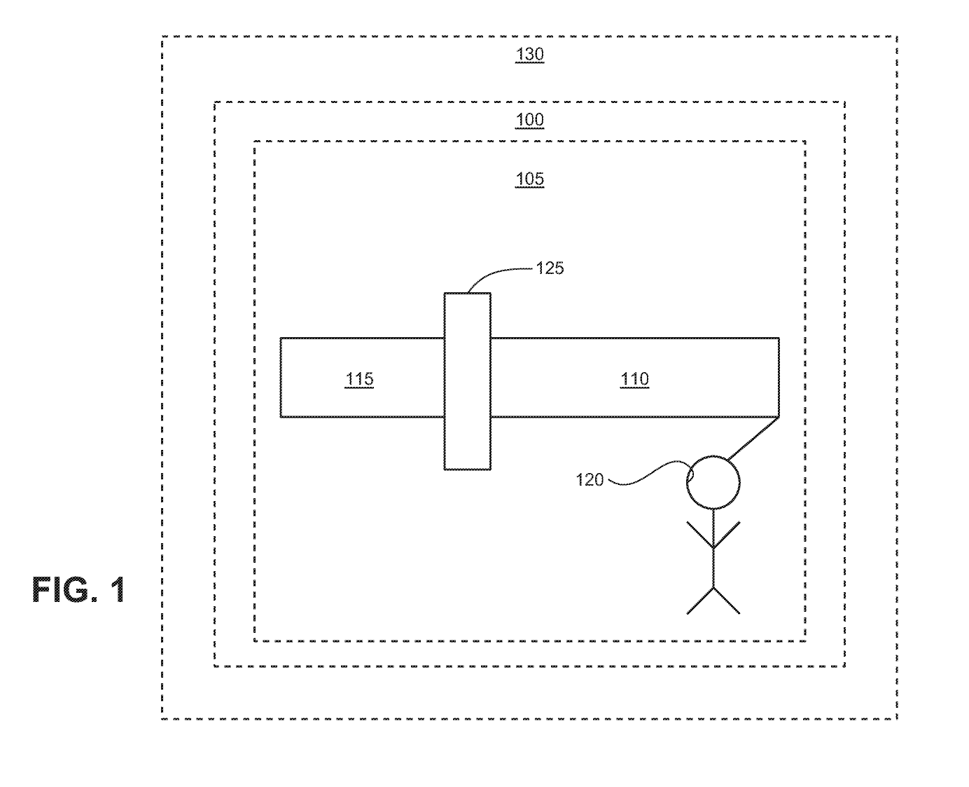

FIG. 1 shows a system 100 for reducing sound level in a respiratory gas delivery system. The system includes a first portion 110 of a breathing circuit tubing 105 and a second portion 115 of the breathing circuit tubing 105 positioned in-line with the first portion 110 of the breathing circuit tubing 105, wherein the second portion 115 of the breathing circuit tubing 105 is a sound-reducing tubing. Hereinafter, the second portion 115 will be referred to as the sound-reducing tubing 115, unless otherwise noted herein. By describing the tubings as being positioned “in-line” with reference to each other, it is meant that the first portion 110 of the breathing circuit tubing 105 is coupled with the second portion 115 of the breathing circuit tubing 105 such that both the first portion 110 and the second portion 115 form at least part of a continuous line of tubing, as part of the breathing circuit tubing 105, through which air may move.

As can be seen in FIG. 1, one end of the first portion 110 is coupled with the patient 120, in one embodiment, while the other end of the first portion 110 is coupled with the second portion 115. The sound-reducing tubing 115 includes features that absorb and/or disrupt the sound waves moving there through, thereby also reducing the sound level. In one embodiment, a generator 205 is also shown to be coupled with the first portion 110.

Thus, the noise leaving the breathing circuit tubing of embodiments is less than that noise leaving the breathing circuit tubing of traditional systems.

In another embodiment, the second portion comprises the first portion 110, such that the sound reducing tubing 115 encompasses the entire length of the breathing circuit tubing 105 (e.g. exhaust tubing).

In one embodiment, the respiratory gas delivery system 130 is a CPAP system. In one embodiment, the CPAP system is an nCPAP system.

In various embodiments, the first portion 110 includes, but is not limited to, at least one of the following: fewer sound-reducing characteristics than the sound-reducing tubing 115; no sound-reducing characteristics; a popple tubing; a flexible material; a correctable material; a compressible material; and a dense material. Further, in one embodiment the first portion 110 is positioned at a patient-end of the breathing circuit tubing 105 while the sound-reducing tubing 115 is positioned at the termination end of the breathing circuit tubing 105.

Additionally, in one embodiment, the first portion 110 is coupled with the sound-reducing tubing 115 via an interference fit. An interference fit refers to a fitting together of components via a physical configuration of the components relative to each other, rather than by a third piece connecting the components to each other. For example, the first portion 110 and the sound-reducing tubing 115 may be shaped such that an end of the sound-reducing tubing 115 snaps within the first portion 110, thereby holding the first portion 110 and the sound-reducing tubing 115 together.

In various embodiments, the sound-reducing tubing 115 at least partially includes, but is not limited to, at least one of the following: a metallocene material; a straight geometry wherein the inside surface of the sound-reducing tubing 115 is smooth; a corrugated geometry; a popple geometry; and at least one ridge that assists in reducing the sound level. For example but not limited to such example, the sound-reducing tubing 115 may be formed of the following combination of materials: 70% polypropylene; and 30% metallocene. Metallocene is an effective sound-reducing material because it is a soft, ductal material that absorbs sound waves. In another embodiment, the sound-reducing tubing 115 may be formed by using a material having similar beneficial sound-reducing properties as metallocene.

It should be noted that the sound-reducing tubing 115 may be of any size facilitating a reduction in sound level and located at any portion of the entire length of the breathing circuit tubing 105. Further, the sound-reducing tubing 115 may be of any length while positioned in-line with the first portion 110.

In one embodiment, the first portion 110 is coupled with the sound-reducing tubing 115 via a connector 125. It should be appreciated that any mechanism that effectively connects the first portion 110 to the sound-reducing tubing 115 without eliminating the benefits described herein of embodiments may be used as the connector 125.

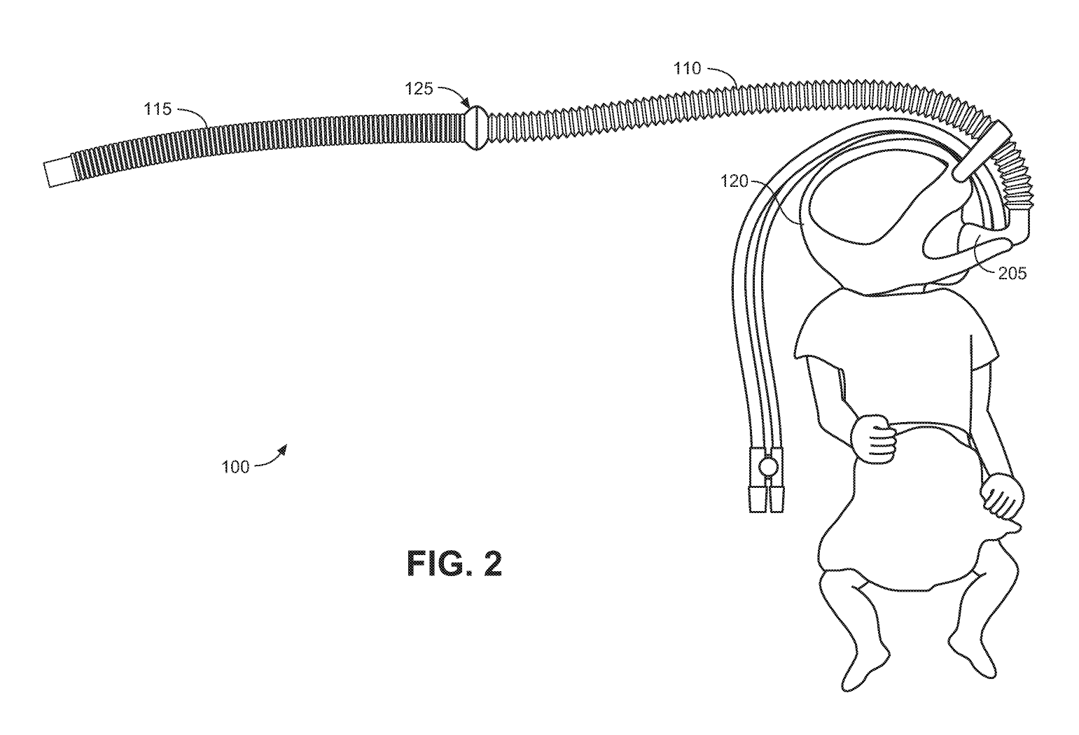

FIG. 2 shows the system 100 for reducing sound level in a respiratory gas delivery system 130, in accordance with embodiments. The first portion 110 is formed of a popple geometry. The sound-reducing tubing 115 is formed at least partially of metallocene.

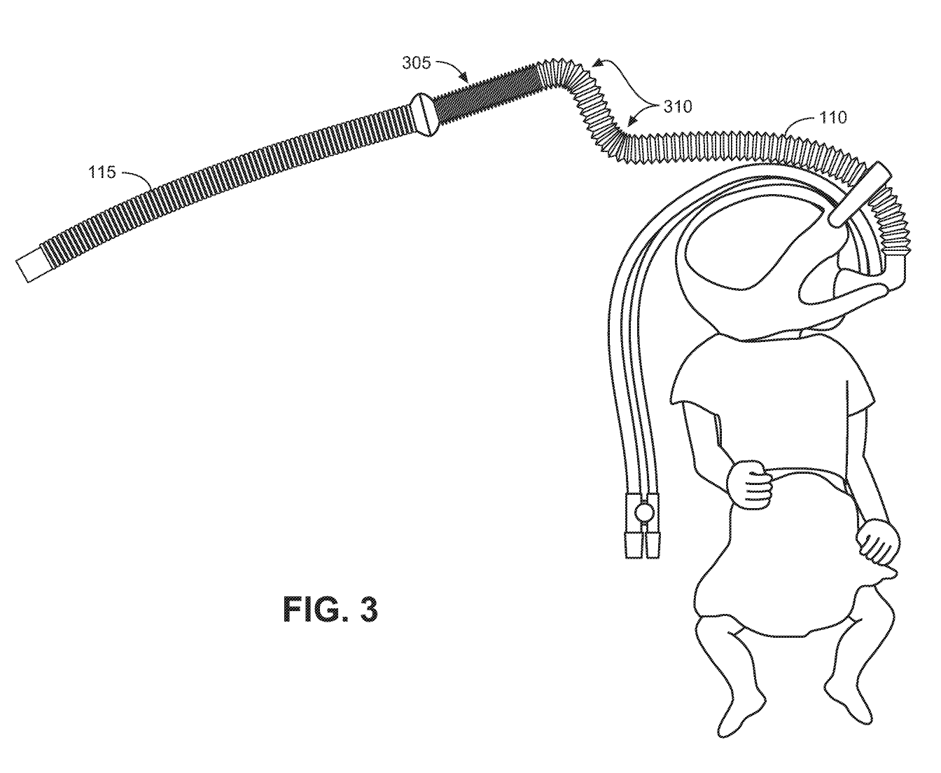

FIG. 3 shows the system 100 for reducing sound level in a respiratory gas delivery system, in accordance with embodiments. More particularly, FIG. 3 shows the first portion 110 including different shapes throughout its length. For example, at 305, a part of the first portion 110 is in a compressed shape. While at 310, another part of the first portion 110 is in a flexed shape. Significantly, the ability of the first portion 110 to be formed into different shapes enables the portion of the breathing circuit that is closest to the patient to be adjusted according to the position of the patient as well as the various components surrounding the patient.

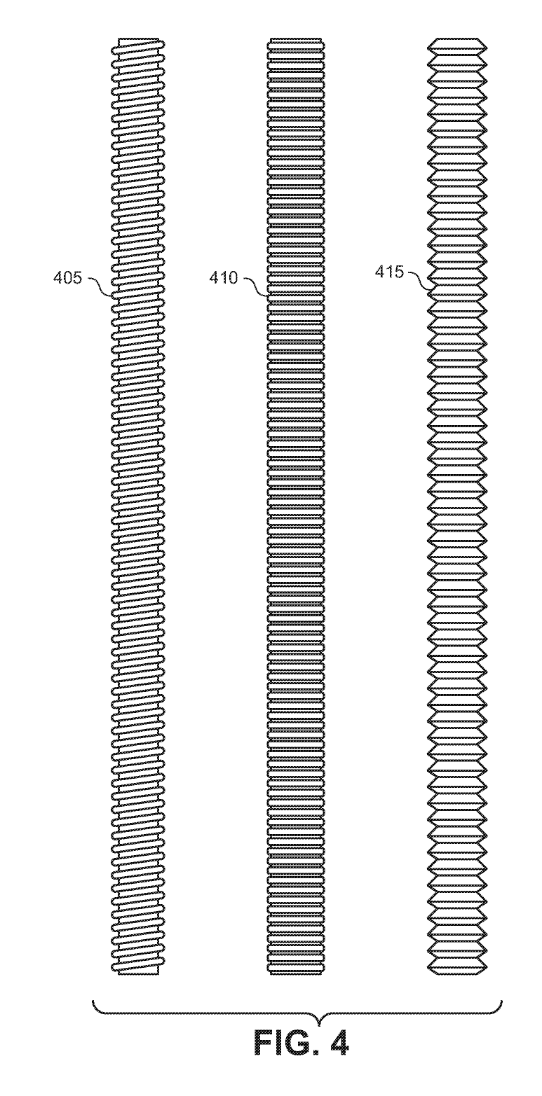

FIG. 4 shows various tubing geometries, in accordance with embodiments. In various embodiments, the tubing geometries may be at least partially included in the first portion 110 and/or the sound-reducing tubing 115. It should be appreciated that while FIG. 4 displays three types of possible tubing geometries, a straight geometry 405, a corrugated geometry 410 and a popple geometry 415, other types of tubing geometries may also be used. The straight geometry 405 has an inside surface that is smooth. The corrugated geometry 410 has ridges on the inside that assist in breaking-up the sound waves and thus reducing the sound level. The popple geometry 415, as described with reference to FIG. 3, may be compressed and flexed at different portions thereof. The popple geometry 415 also has ridges on the inside that break up the sound waves. Of the three different types of geometries described, the popple geometry 415 provides the greatest sound-reducing effects.

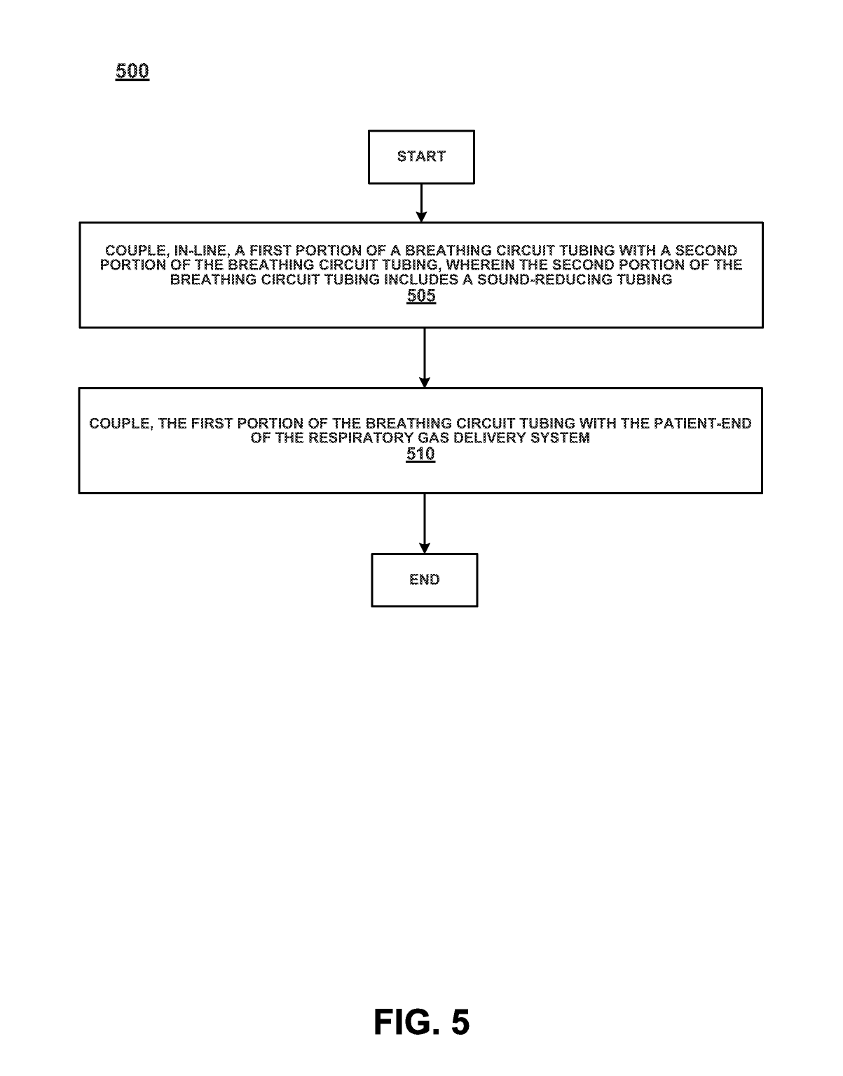

FIG. 5 shows a flow chart 500 of an example method for manufacturing a system for reducing sound level in a respiratory gas delivery system, in accordance with embodiments. With reference to FIGS. 1-5 and as described herein, in one embodiment, at step 505, the first portion 110 of the breathing circuit tubing 105 is coupled in-line with a second portion 115 of the breathing circuit tubing 105, wherein the second portion 115 comprises a sound-reducing tubing. In one embodiment and as described herein, the sound-reducing tubing 115 is at least partially formed from metallocene material. In another embodiment and as described herein, the sound-reducing tubing 115 includes at least one ridge. The at least one ridge may be on the inside surface and/or the outside surface of the sound-reducing tubing 115.

At 510, in one embodiment and as described herein, the method 500 further includes coupling the first portion 110 of the breathing circuit tubing 105 with the patient-end of the respiratory gas delivery system 130.

All statements herein reciting principles, aspects, and embodiments of the present technology as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present technology, therefore, is not intended to be limited to the embodiments shown and described herein. Rather, the scope and spirit of present technology is embodied by the appended claims.

What is claimed is:

1. A system for reducing sound level in a respiratory gas delivery system, said system comprising:
a first portion of a breathing circuit tubing; and
a second portion of said breathing circuit tubing positioned in-line with said first portion of said breathing circuit tubing, wherein said second portion of said breathing circuit tubing is a sound-reducing tubing.

2. The system of claim 1, wherein said respiratory gas delivery system comprises:
a continuous positive airway pressure (CPAP) system.

3. The system of claim 2, wherein said CPAP system comprises:
an infant nasal CPAP (nCPAP) system.

4. The system of claim 1, wherein said first portion of said breathing circuit tubing comprises:
fewer sound-reducing characteristics than said sound-reducing tubing.

5. The system of claim 4, wherein said first portion of said breathing circuit tubing does not comprise sound-reducing characteristics.

6. The system of claim 1, wherein said first portion of said breathing circuit tubing is part of a single-limb respiratory circuit.

7. The system of claim 1, wherein said first portion of said breathing circuit tubing comprises:
a popple tubing.

8. The system of claim 1, wherein said first portion of said breathing circuit tubing is positioned at a patient-end of said breathing circuit tubing and said sound-reducing tubing is positioned at a termination end of said breathing circuit tubing, wherein said termination end is opposite said patient-end.

9. The system of claim 1, wherein said first portion of said breathing circuit tubing comprises:
a flexible material.

10. The system of claim 1, wherein said sound-reducing tubing at least partially comprises:
a metallocene material.

11. The system of claim 1, wherein said sound-reducing tubing comprises:
a straight geometry, wherein an inside surface of said sound-reducing tubing is smooth.

12. The system of claim 1, wherein said sound-reducing tubing comprises:
a corrugated geometry.

13. The system of claim 1, wherein said sound-reducing tubing comprises:
a popple geometry.

14. The system of claim 1, wherein said sound-reducing tubing comprises:
at least one ridge.

15. The system of claim 1, wherein said first portion of said breathing circuit tubing is coupled with said sound-reducing tubing via an interference fit.

16. The system of claim 15, wherein said interference fit comprises:
a snapping feature, wherein an end of one of said first portion of said breathing circuit tubing and said sound-reducing tubing snaps within an end of the other of said first portion of said breathing circuit tubing and said sound-reducing tubing.

17. The system of claim 1, wherein said first portion of said breathing circuit tubing is coupled with said sound-reducing tubing via a connector.

18. The system of claim 1, wherein said second portion of said breathing circuit tubing comprises said first portion.

19. A method for manufacturing a system for reducing a sound level in a respiratory gas delivery system, said method comprising:
coupling, in-line, a first portion of a breathing circuit tubing with a second portion of said breathing circuit tubing, wherein said second portion of said breathing circuit tubing comprises a sound-reducing tubing.

20. The method of claim 19, wherein said coupling further comprises:
coupling said first portion of said breathing circuit tubing with a patient-end of said respiratory gas delivery system.

21. The method of claim 19, wherein said coupling comprises:
coupling, in-line, said first portion of said breathing circuit tubing with said sound-reducing tubing, wherein said sound-reducing tubing is formed at least partially from metallocene material.

22. The method of claim 19, wherein said coupling comprises:
coupling, in-line, said sound-reducing tubing that comprises at least one ridge.
